(19) United States
(12) Patent Application Publication
(10) Pub. No.: US 2026/0002184 A1
(43) Pub. Date: Jan. 1, 2026

(54) METHOD FOR PRODUCING MASSOIA LACTONE FROM LIAMOCINS

(71) Applicant: The United States of America, as represented by the Secretary of Agriculture, Washington, DC (US)

(72) Inventor: EVAN C. WEGENER, GERMANTOWN HILLS, IL (US)

(21) Appl. No.: 19/247,573

(22) Filed: Jun. 24, 2025

Related U.S. Application Data
(60) Provisional application No. 63/664,766, filed on Jun. 27, 2024.

(51) Int. Cl. C12P 17/06; C07D 309/30

(52) U.S. Cl. CPC C12P 17/06; C07D 309/30

(57) ABSTRACT

Disclosed herein are methods of producing Massoia lactone from liamocins, or mixtures of liamocins and exophilins. Liamocins utilizable in the methodologies provided herein can be produced by microbes, such as Aureobasidium pullulans. The methodologies described herein utilize acid catalyzed hydrolysis/dehydration and carboxylic acids as a phase transfer agent in a dual-solvent system. This approach further allows the separation and collection of liamocin head groups.

### CROSS-REFERENCE

The present application claims priority to U.S. Provisional Patent Application Ser. No. 63/664,766 filed Jun. 27, 2024, the contents of which are expressly incorporated herein by reference.

### BACKGROUND OF THE INVENTION

### Field of Invention

Disclosed herein are methods of producing Massoia lactone from liamocins, or mixtures of liamocins and exophilins. Liamocins utilizable in the methodologies provided herein can be produced by microbes, such as Aureobasidium pullulans. The methodologies described herein utilize acid catalyzed hydrolysis/dehydration and carboxylic acids as a dual catalyst and phase transfer agent in a mixed solvent system. This approach further allows the separation and collection of liamocin head groups.

### Background

Massoia lactone (5-hydroxy-2-decenoic acid lactone, or “ML”) is a C₁₀ δ-lactone commonly used as a flavoring and fragrance agent due to its aroma that is described as coconutty or buttery. It has also been reported to have an array of therapeutic properties including antifungal, antibacterial, and anticancer activity (Wang et al, World J. Microbiol. Biotech., (2022), 38:107; Zhang et al, Microbiol. Res., (2018), 242:126641; Barros et al, Eur. J. Med. Chem., (2014), 76:291-300). Several pathways have been developed (Mineeva, I., Russ. J. Org. Chem., (2012), 48:977-81; Harbindu & Kumar, Synthesis, (2011), 12:1954-9; Bennett et al., J. Chem. Soc., (1991), 6:1543-7) to synthesize ML which has traditionally been isolated from the bark of the Cryptocarya massoi tree native to Southeast Asia. Due to its myriad of potential uses, more sustainable routes for producing ML from renewable feedstocks have also been sought. One potential source of ML are the extracellular oils produced by certain strains of Aureobasidium during biomass fermentation processes (Wan et al, Biochem. Eng. J., (2022), 188:108687; Kurosawa et al, Biosci. Biotech. Biochem., (1994), 11:2057-60). These oils, named liamocins, consist of a polyol head group esterified to a tail of three or four 3,5-dihydroxydecanoic acid molecules connected through the hydroxyl group on the fifth carbon of the preceding acid (Price et al, Carbohydrate Res., (2013), 370:24-32).

Although the potential uses of biobased, sustainably sourced ML have been widely studied, less work has been done on developing scalable green methods for converting liamocins to this versatile product. The formation of ML from liamocins by refluxing the oil in methanolic hydrochloric acid has previously been reported (Price et al, (2013), supra). Similarly, δ-lactone of 3,5-dihydroxydecanoic acid, isolated from a culture of Cephalosporium recifei, was dehydrated using para-toluene sulfonic acid in boiling benzene (Vesonder et al, Can. J. Biochem., (1972), 50:363-5). Recently a multi-step process has been routinely used in lab scale preparations of ML from liamocins. First an homogeneous base is used to catalyze hydrolysis of the oil in an aqueous solution. This is followed by acidification with an aqueous mineral acid to neutralize the base and catalyze the dehydration reaction. ML lactone is then extracted from the reaction mixture using dichloromethane (Tang et al, Process Biochem., (2018), 69:64-74). While effective for obtaining ML at the laboratory scale, these methods have numerous short comings with respect to developing a scalable green process such as the use of corrosive catalysts, generation of waste through sequential basification/acidification, and use of hazardous solvents. Additionally, using either pure water or an organic solvent as the reaction medium may cause difficulties due to the emulsifying properties of liamocins (Manitchotpisit et al, Biotech. Lett., (2011), 33:1151-7) in the former and insolubility of the polyol head group in the latter. The instant disclosure provides details on the development of an easily scalable and environmentally friendly process for producing Massoia lactone from liamocins.

### SUMMARY OF THE INVENTION

The disclosure herein provides a method of producing Massoia lactone, by first preparing a reaction mixture of water, a carboxylic acid, methyl ethyl ketone, and a liamocin; and incubating the reaction mixture under conditions allowing for the conversion of the liamocin to Massoia lactone. In some embodiments of this method, the ratio of water to carboxylic acid is about 20:80 to about 80:20. In exemplary embodiments, the carboxylic acid is succinic acid, malic acid, tartaric acid, citric acid, or a combination thereof. In some embodiments, the reaction mixture further comprises an exophilin. In such embodiments, the conditions also allow for the conversion of the exophilin to Massoia lactone. In some embodiments, the water and carboxylic acid are combined prior to adding to the reaction mixture. In some embodiments, the methyl ethyl ketone and liamocin are combined prior to adding to the reaction mixture. In some embodiments, the liamocin comprises an Aureobasidium pullulans culture filtrate. In some embodiments, the incubating step is performed for at least about one hour. In some embodiments, the reaction conditions allow for the conversion of liamocin to a mixture of a hydrated lactone and Massoia lactone.

### INCORPORATION BY REFERENCE

All publications, patents and patent applications mentioned in this specification are herein incorporated by reference to the same extent as if each individual publication, patent or patent application was specifically and individually indicated to be incorporated by reference.

### BRIEF DESCRIPTION OF THE DRAWINGS

The novel features of the invention are set forth with particularity in the claims. Features and advantages of the present invention are referred to in the following detailed description, and the accompanying drawings of which:

FIG. 1 provides a chemical structural drawing of a liamocin and an exophilin.

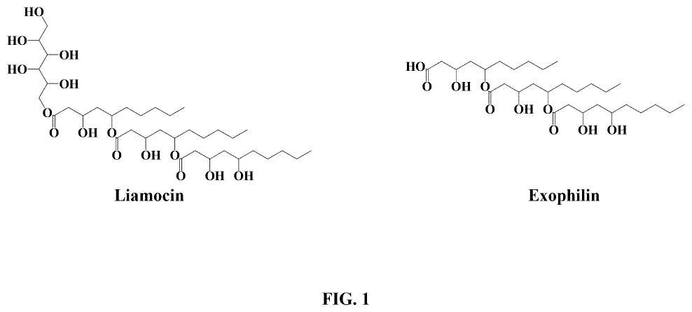

FIG. 2 provides a representation of the chemical conversion of liamocins to Massoia lactone through a reaction cascade which can be catalyzed by carboxylic acids in a dual solvent system (MEK, water). The chemical conversion route is also possible with exophilins which can be present in the oil feed or produced from liamocins by hydrolysis between the polyol head and fatty acid tail.

FIG. 3 provides an exemplary process flow diagram for conversion of liamocins to Massoia lactone and collection of Massoia lactone and head group products.

FIG. 4 provides a ternary liquid-liquid equilibrium diagram for MEK/water/citric acid system at 24° C. The solid and open diamonds represent the organic and aqueous rich phases, respectively as determined from the composition of biphasic mixtures. The binodal curve, as determined from monophasic mixtures by titration with MEK and water, is represented by the solid and open circles, respectively. The solid squares represent the saturation concentration of citric acid in MEK and water.

FIG. 5 provides representation of the effect of citric acid loading on the yield of Massoia lactone versus time. Values in legend refer to the mass fraction of citric acid and H₂O. The relative amounts of citric acid and H₂O were changed while the total amount of citric acid and H₂O was held constant (combined mass fraction=0.50). Reactions were performed at a fixed total reaction mass (25 g) and MEK (0.45) and liamocin (0.05) mass fractions. Squares—18 wt % citric acid/32 wt % water, circles—22 wt % citric acid/28 wt % water, and triangles—27 wt % citric acid/23 wt % water.

FIG. 6 provides graphical representation of the effect of water content on the yield of Massoia lactone versus time. Values in legend refer to the mass fraction of H₂O and MEK The relative amounts of H₂O and MEK were changed while the total amount of H₂O and MEK was held constant (combined mass fraction=0.77). Reactions were performed at a fixed total reaction mass (25 g) and citric acid (0.18) and liamocin (0.05) mass fractions. Squares—52 wt % water/25 wt % MEK, circles—32 wt % water/45 wt % MEK, and triangles—12 wt % water/65 wt % MEK.

FIG. 7 provides graphical representation of the effect of liamocin content on the yield of Massoia lactone versus time. Values in legend refer to the initial mass fraction of liamocin. The relative amounts of citric acid, H₂O, and MEK were held constant at a citric acid:H₂O:MEK mass ratio=1.0:1.8:2.5 while the total reaction mass was increased with the addition of more oil. The citric acid mass fraction of the liamocin free system=0.18. Squares—5 wt % liamocins, circles—7.5 wt % liamocins, and triangles—10 wt % liamocins.

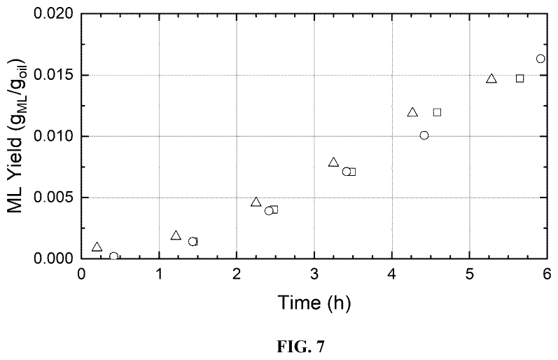

FIG. 8 provides a ternary liquid-liquid equilibrium diagram for MEK/water/L-tartaric acid system at 24° C. The solid and open diamonds represent the organic and aqueous rich phases, respectively. Solid triangles represent feed compositions which did not phase separate.

FIG. 9 provides a ternary liquid-liquid equilibrium diagram for MEK/water/L-malic acid system at 24° C. The solid and open diamonds represent the organic and aqueous rich phases, respectively. Solid triangles represent feed compositions which did not phase separate.

FIG. 10 provides a ternary liquid-liquid equilibrium diagram for MEK/water/L-succinic acid system at 24° C. The solid and open diamonds represent the organic and aqueous rich phases, respectively. Solid triangles represent feed compositions which did not phase separate.

FIG. 11 provides graphical representation of Massoia lactone yield as a function of time using different carboxylic acids as phase transfer agents and catalysts. Reactions were performed at a fixed total reaction mass (25 g) and MEK (0.45) and liamocin (0.05) mass fractions. The combined acid and water mass fraction was fixed at 0.5, but the relative amounts of these were adjusted to maintain a constant acid loading of 1.27 mmol acid/g_{reaction mixture}. C.A.=citric acid, T.A.=L-tartaric acid, M.A.=L-malic acid, and S.A.=succinic acid. Squares—1.27 mmol C.A./g, circles—0.85 mmol C.A./g, triangles—1.27 mmol T.A./g, diamonds—1.27 mmol/g M.A., and stars—1.27 mmol/g S.A.

FIG. 12 provides graphical representation of Massoia lactone yield as a function of residence time in a plug flow reactor. Reactions were performed with a feed composition with mass fractions of citric acid (0.25), water (0.25), MEK (0.45), and liamocins (0.05). Reactions were performed at 500 psi and either 120° C. (circle) or 150° C. (squares).

### DETAILED DESCRIPTION OF THE INVENTION

Herein disclosed is a scalable and environmentally friendly process for producing Massoia lactone (ML) from liamocins (also termed liamocin oils). In some embodiments, mixtures of liamocins and exophilins, such as from cultures of A. pullulans, are utilized for the methods provided herein. Methods of the present disclosure preferably utilize non-toxic carboxylic acids, which can be produced by fermentation, to catalyze sequential hydrolysis-dehydration reactions. In one embodiment of the methods disclosed herein, water and methyl ethyl ketone (MEK) were used as co-solvents to solubilize all reactants and products and the methodology exhibited unexpected phase-equilibria behavior when combined with the carboxylic acids. Non-toxic carboxylic acids, which can be produced by fermentation, are preferably used to catalyze the sequential hydrolysis-dehydration reactions. In some embodiments, the use of water and MEK (also termed butanone, and 2-butanone) as co-solvents was found to be effective for solubilizing all reactants and products and exhibited unexpected phase-equilibria behavior when combined with the carboxylic acids. The methods disclosed herein can be performed under multiple pressure and temperature points, at small or large scale, and in batch or flow reactors.

Water and MEK are only partially miscible (forming an aqueous phase with 23 wt % MEK and an organic phase that is 12.5 wt % water). Additionally, carboxylic acids (e.g., citric acid, malic acid) are only sparingly soluble in MEK. Surprisingly, addition of aqueous acids increases the mutual solubilities of the two solvents (water and MEK), finally reaching total miscibility at high enough concentrations.

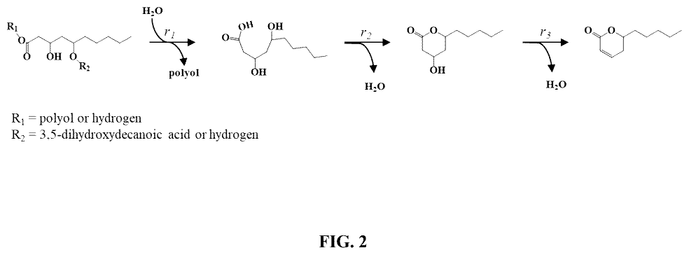

Multiple additional parameters (e.g., temperature and pressure) were tested, allowing the development of a process flow diagram exemplary of potential scale-up. Liamocins can be converted to ML through a series of reactions, as depicted in FIG. 2. As illustrated by this reaction, liamocin oil is hydrolyzed to release the polyol head group and form 3,5-dihydroxydecanoic acid (r₁). The free acid readily forms the δ-lactone (r₂) and then undergoes dehydration to form ML (r₃).

Preferred embodiments of the present invention are shown and described herein. It will be obvious to those skilled in the art that such embodiments are provided by way of example only. Numerous variations, changes, and substitutions will occur to those skilled in the art without departing from the invention. Various alternatives to the embodiments of the invention described herein may be employed in practicing the invention. It is intended that the included claims define the scope of the invention and that methods and structures within the scope of these claims and their equivalents are covered thereby.

Technical and scientific terms used herein have the meanings commonly understood by one of ordinary skill in the art to which the instant invention pertains, unless otherwise defined. Reference is made herein to various materials and methodologies known to those of skill in the art.

Any suitable materials and/or methods known to those of skill can be utilized in carrying out the instant invention. Materials and/or methods for practicing the instant invention are described. Materials, reagents and the like to which reference is made in the following description and examples are obtainable from commercial sources, unless otherwise noted. This disclosure teaches methods and describes tools for producing Massoia lactone from liamocin-containing oils. In some embodiments, these methods can also be utilized to produce liamocin head groups (e.g., mannitol, glycerol).

As used in the specification and claims, use of the singular “a”, “an”, and “the” include plural references unless the context clearly dictates otherwise.

The terms isolated, purified, or biologically pure as used herein, refer to material that is substantially or essentially free from components that normally accompany the referenced material in its native state.

The term “about” is defined as plus or minus ten percent of a recited value. For example, about 1.0 g means 0.9 g to 1.1 g and all values within that range, whether specifically stated or not.

### Liamocins

Methodologies of the instant disclosure can be practiced using any liamocin or liamocin oil. Exemplary liamocins include, but are not limited to, glycerol-liamocins, threitol-liamoicins, erythritol-liamocins, mannitol-liamocins, arabitol-liamocins, 2-amino-D-mannitol liamocins, 2-N-acetylamino-D-mannitol liamocins, D-fucitol liamocins, fructose-liamocins and L-rhamnitol liamocins. Liamocins can be derived from any source, including cultures of Aureobasidium pullulans.

The instant methodologies can also be utilized to produce Massoia lactone from exophilins (FIG. 2). Culture filtrates of A. pullulans can contain both liamocins and exophilins. Exophilins are defined as oligomers of 3,5-dihydroxydecanoic, esterified through the hydroxyl group on carbon five, which are lacking a polyol head group on the first carboxylic acid group. (Price et al, (2013), supra) (Price et al, Carbohydrate Res., (2013), 370:24-32)

### Process Flow Diagram

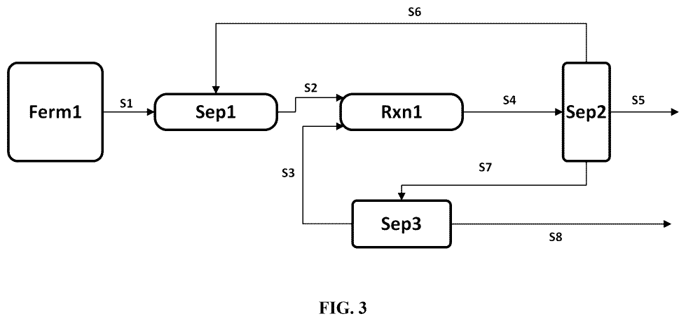

Provided herein is an exemplary process for the production of ML from liamocins/liamocin oils (FIG. 3). The skilled artisan will understand that this process flow diagram can be modified to achieve the end product(s) desired and will recognize that the chemistry of recovering one head group product may differ from the chemistry of recovering other head groups. In the process flow diagram shown in FIG. 3, “Ferm1” is a fermenter for producing liamocin oils. This fermenter can be an actively growing culture of, for example, A. pullulans. “Sep 1” indicates the separation of oils from cells/fermentation broth, which can be achieved via centrifugation or extraction with MEK. “Rxn1” is a reactor for sequential hydrolysis-dehydration of liamocins to Massoia lactone in the presence of water, MEK and a carboxylic acid. Any reactor known in the art, such as a plug flow reactor (PFR) or continuous stirred tank reactor (CSTR) can be utilized. “Sep 2” indicates the removal of MEK in which the resultant liquid phase undergoes spontaneous phase separation. The top organic layer is the organic phase containing Massoia lactone and 3-hydroxydecanoic acid lactone and bottom layer is the aqueous phase containing the acid catalysts and liamocin head group. Separation could be achieved, for example, by flash distillation with a vapor-liquid-liquid separation or conventional distillation where the liquid-liquid separation happens in the reboiler. “Sep3” indicates the recovery of the liamocin head group (e.g., mannitol) from the aqueous catalyst solution. Separation could be achieved using methods such as crystallization or chromatography. FIG. 3 also shows the following exemplary process stream descriptions. “S1” denotes the recovery of liamocins/liamocin oil from a microbial fermentation broth. “S2” denotes combination of liamocin oil and MEK, which is then fed into the hydrolysis-dehydration reactor. “S3” denotes the catalyst/water recycling after recovery of the liamocin head group. “S4” denotes the hydrolysis-dehydration reactor effluent. “S5” denotes the Massoia lactone and 3,5-dihydroxydecanoic acid product stream. “S6” denotes the recycling of MEK. “S7” denotes the stream containing the liamocin head group in water. “S8” denotes the production stream for the liamocin head group product or head group waste stream where recovery of the head group is not desired. An alternative recovery method to that shown in FIG. 3 could be to separate the carboxylic acid from the effluent of “Rxn1” and cause phase separation of the organic phase, rich in ML, and the aqueous phase. ML could then be recovered from MEK by distillation, for example. The skilled artisan will understand various permutations of this exemplary embodiment can be designed and still practice the methodologies disclosed herein.

### Reaction Parameters

Reactions of the instant disclosure can be performed at various pressures and temperatures (e.g., atmospheric pressure, ambient temperature), but can be selected to maximize product output, minimize cost, ease of separation of end-products, or a combination of these. Other parameters include the ratio of MEK:water which can be from 80:20 to 20:80, or any individual ratio between these two points. In some embodiments an MEK:water ratio of 50:50 is utilized. Temperatures at which the reactions of the instant disclosure are performed usually will be below the temperature at which the organic acid(s) degrades. Pressures utilized will typically keep reaction and products condensed and acid concentration can be adjusted so as to maintain a single phase. The reactions and methodologies provided herein can be performed in monophasic or biphasic systems. In some embodiments, the reaction conditions allow for the conversion of liamocin to a mixture of a hydrated lactone and Massoia lactone. Such hydrated lactone can be the majority product in a given reaction.

### Carboxylic Acids

Carboxylic acids are known in the art and the carboxylic acids useful in practicing the reactions of the instant disclosure include, but are not limited to: citric acid, L-tartaric acid, L-malic acid, succinic acid, acetic acid, oxalic acid, malonic acid, maleic acid, fumaric acid, itaconic acid, citramalic acid, adipic acid, glucaric acid, propionic acid, butyric acid, lactic acid, and pyruvic acid. Reactions of the instant disclosure can be performed utilizing a single carboxylic acid, or can be performed with mixtures of 1, 2, 3, 4, 5, or more carboxylic acids. Some non-limiting examples of acid combinations include combinations of citric acid, tartaric acid, malic acid, and succinic acid in any combination.

In preferred embodiments, the reactions and methodologies provided herein do not utilize corrosive acids like hydrochloric acid or sulfuric acid. In preferred embodiments, the reactions and methodologies do not use carcinogenic solvents such as benzene or dichloromethane.

The embodiments illustratively disclosed herein suitably may be practiced in the absence of any element [e.g., method (or process) steps or composition components)] which is not specifically disclosed herein. Thus, the specification includes disclosure by silence. Written support for a negative limitation may also be found through the absence of the excluded element in the specification, known as disclosure by silence.

Having generally described this invention, the same will be better understood by reference to certain specific examples, which are included herein to further illustrate the invention and are not intended to limit the scope of the invention as defined by the claims.

### EXAMPLES

### Example 1

### Phase Diagram Construction

Liquid-liquid equilibrium phase diagrams were constructed by determining the MEK and acid concentrations of the organic and aqueous phases formed from a series of ternary mixtures of known bulk compositions. Feed solutions were prepared in 1.5-dram vials using equal masses of water and MEK (1.83-2.00 g) and the quantity of acid needed for a total mass of 4.0 g. The carboxylic acid was dissolved in water and then MEK was added followed by vigorous shaking for ≈2 min. The vials were allowed to equilibrate for 24 hr at room temperature (298.5±1 K) prior to compositional analysis. The MEK and acid concentrations in each phase was determined using a Shimadzu HPLC equipped with a Bio-Rad 87-H column. 5 mM sulfuric acid in water was used as the mobile phase with a flow rate of 0.5 mL/min and column temperature of 65° C. MEK and the carboxylic acids were measured using a prominence diode array detector (PDA) at wavelengths of 210 and 265 nm, respectively. Response factors for each compound were determined from calibrations curves of reference samples of known concentrations. Samples were prepared for analysis by diluting 30 mg of the equilibrated organic/aqueous phase in 1 g of mobile phase.

### Liamocin Conversion in Batch Reactors

Initial liamocin conversion experiments were performed at 70° C. and atmospheric pressure in batch reactors consisting of 50 mL three-neck round bottom flask charged with a Teflon coated stir bar and fixed with a water cooler condenser, a thermocouple port, and a rubber septum. Reactors were heated in an aluminum bead bathed seated on a stirring hot plate and the temperature was controlled using a probe positioned in the reaction mixture. The organic (liamocin oil in MEK) and aqueous (carboxylic acid in water) feeds were prepared in in separate scintillation vials and shaken until the oil/acid was fully dissolved. All reactions were performed with a constant total feed mass of 25 g. Reactions were started by adding the two feeds to the reaction flask followed by immediately turning on the heating and stirring (400 rpm). To monitor the progress of the reactions, samples (300 μL) were withdrawn through the rubber septum using a needle and syringe. Samples were taken upon introduction of the two feeds, once the reaction reached 70° C., and then every hour.

The formation of Massoia lactone was monitored with a Shimadzu HPLC equipped with a C18 Column (Phenomenex: Luna 5μ C18 (2) 100 Å, 250×4.6 mm) held at 35° C. using a PDA with a detection wavelength of 210 nm. Massoia lactone was quantified using a calibration curve constructed from references samples of known concentrations. Reaction samples were diluted in a mixture of 50% 5 mM sulfuric acid in water/50% acetonitrile. A solvent gradient with 5 mM sulfuric acid in water and acetonitrile was used for elution and the total flow rate was held constant at 0.7 mL/min. The gradient method employed consisted of flowing 90% 5 mM sulfuric acid/water-10% acetonitrile for 3 min, a linear ramp to 82% acetonitrile over 20 min, holding at 82% acetonitrile for 10 min, and then a linear ramp to 90% 5 mM sulfuric acid in water over 15 min.

Hydrolysis-dehydration reactions run for 24 hr were performed in 1 mL Reacti-Vials. The acid catalyst, water, and organic feed (10 wt % Liamocin oil in MEK) were added to the vials with a Teflon spin vane. The vials were capped with Teflon-backed septa and placed into an aluminum heating block preheated to 70° C. on a stirring hot plate. After 24 hr the vials were removed from the heating block, cooled in an ice bath, and analyzed using the HPLC method described above.

### Results

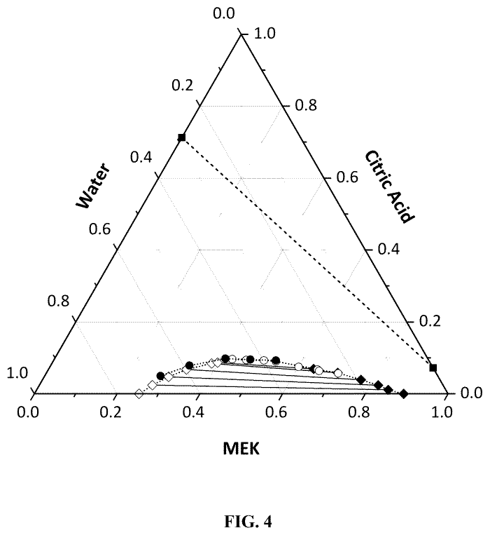

Water and MEK are immiscible, when mixed the two species partition in to an organic phase (89% MEK by mass) and an aqueous phase (75% H₂O by mass). Attempts of using citric acid as a catalyst for the hydrolysis-dehydration of liamocins revealed citric acid increases the mutual solubility of MEK and H₂O. Based on this finding, a liquid-liquid phase diagram (FIG. 4) was constructed at 24° C. by letting mixtures of equal masses of MEK and H₂O with varying amount of citric acid equilibrate overnight.

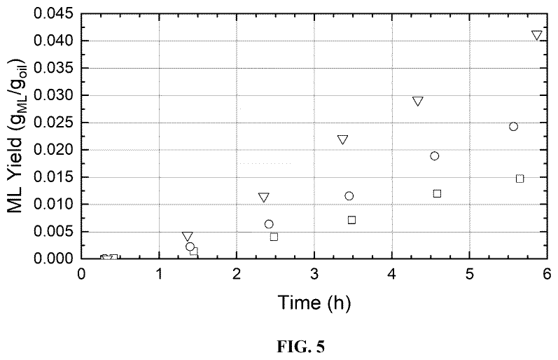

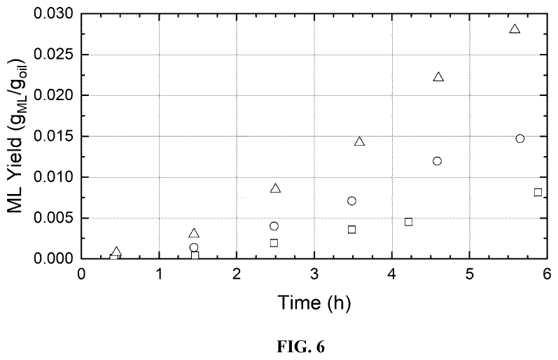

Increasing the mass fraction of citric acid in the mixture increases the mutual solubility of MEK in H₂O, and vice versa, and the two components becoming completely miscible above ≈10% citric acid. Based on the developed phase diagram, liamocin hydrolysis-dehydration reactions were performed in the ternary system at citric acid concentrations high enough to produce mono-phasic systems [>10 wt %]. Results for reactions conducted at 70° C. with varying amounts of citric acid are shown in FIG. 5 and there is an observed increase in Massoia lactone yield at higher acid concentration. Interestingly, we noted a decrease in Massoia lactone yield at higher water concentrations (FIG. 6). The concentration of liamocin appears to have a negligible effect on the Massoia lactone yield. No solid precipitates or phase separation were observed of the course of these reactions.

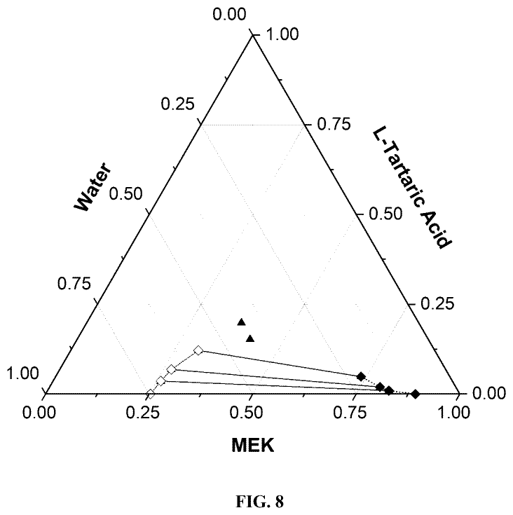

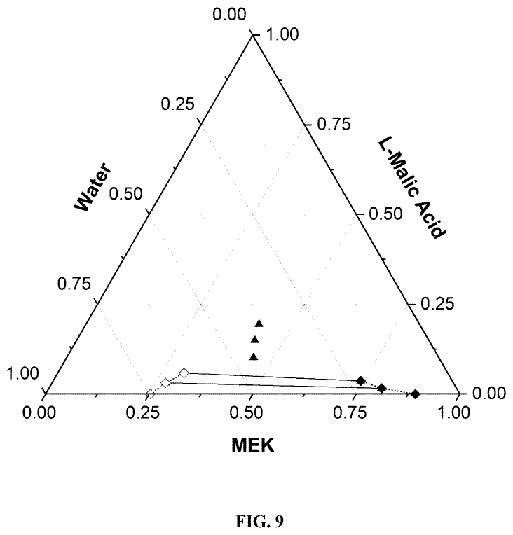

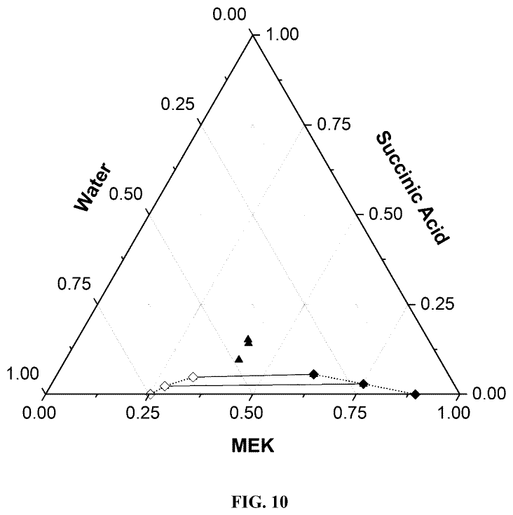

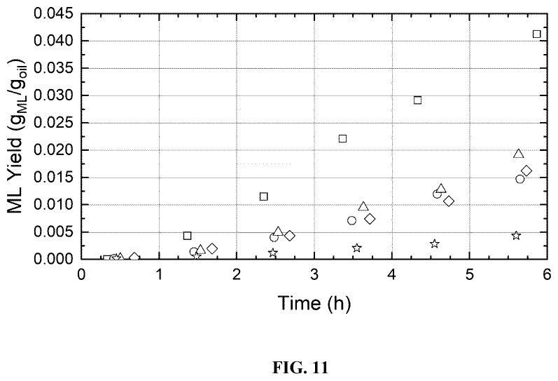

Other carboxylic acids which are naturally occurring and/or can be produced via fermentation were also tested to see whether they exhibited the same phase transfer/catalytic capabilities of citric acid in the MEK-water system. Ternary phase diagrams were constructed for L-tartaric acid, L-malic acid, and succinic acid at 24° C. by letting mixtures of equal masses of MEK and H₂O with varying amount of acid equilibrate overnight (FIG. 8, FIG. 9, FIG. 10). All three acids showed similar behavior to citric acid with respect to increasing the mutual solubility of MEK and water. Additionally, all make the two components completely miscible around 10 wt %. As the acids become more non-polar (i.e., lose hydroxyl groups) they less selectively partition into the aqueous phase of mixtures that phase separate. Like with citric acid, catalytic reactions were performed at 70° C. using acid concentrations which produced mono-phasic mixtures at 24° C. (FIG. 11).

Unlike citric acid, which is a triacid, the other carboxylic acids tested here were diacids. For the diacids, a constant loading of 1.27 mmol_{acid}/g_{reaction mixture} was used for the hydrolysis-dehydration reactions. Citric acid was tested at two different loadings: 1.27 mmol/g to match the molecular concentrations of the diacids and 0.85 mmol/g which gives an equivalent concentration of carboxylic acid groups to the diacids. Of all the acids tested, citric acid produced the highest yield of Massoia lactone for 1.27 mmol/g citric acid. 0.85 mmol/g citric acid, 1.27 mmol/g tartaric acid, and 1.27 mmol/g malic acid give similar yields which are approximately 2.2-2.7 times lower than that of 1.27 mmol/g citric acid after 5.5 hours. 1.27 mmol/g succinic acid gives the lowest yields of Massoia lactone, approximately 3-4 times lower than 0.85 mmol/g citric acid, 1.27 mmol/g tartaric acid, and 1.27 mmol/g malic acid. These results indicate that all acids tested here can act as phase transfer agents and catalysts.

Reactions were also run with different concentrations of citric, tartaric, malic and succinic acid, and the other acids for 24 hr to determine yields of Massoia lactone under the conditions tested above and the results are in Table 1. No solid precipitates were seen in any of these tests. As with shorter times, the highest yield of Massoia lactone (0.152 g/g_{liamocin}) is seen with citric acid at a loading of 1.29 mmol_{acid}/g_{reaction mixture}. The maximum lactone yield assuming 2.5 dihydroxydecanoic acids/liamocin would be 0.72 g/g_{liamocin}. Results here for the unoptimized reaction corresponds to 21% of this estimated maximum.

TABLE 1
Massoia lactone yields from reaction performed at 70° C. for 24 hr.

| | | | | | | Massoia |
|---|---|---|---|---|---|---|
| | | | | | Acid Loading | Lactone |
| | Reaction Mixture Mass Fractions | | | | (mmol/ | Yield |
| Acid | Acid | Water | MEK | Liamocin | g_{reaction mixture}) | (g/g_{Liamocin}) |
| Citric Acid | 0.25 | 0.25 | 0.45 | 0.05 | 1.29 | 0.143 |
| Citric Acid | 0.20 | 0.30 | 0.45 | 0.05 | 1.06 | 0.103 |
| Citric Acid | 0.16 | 0.34 | 0.45 | 0.05 | 0.85 | 0.068 |
| Tartaric Acid | 0.19 | 0.31 | 0.45 | 0.05 | 1.28 | 0.063 |
| Malic Acid | 0.17 | 0.33 | 0.45 | 0.05 | 1.28 | 0.052 |
| Succinic Acid | 0.15 | 0.35 | 0.45 | 0.05 | 1.28 | 0.014 |
| Sulfuric Acid | 0.13 | 0.37 | 0.45 | 0.05 | 1.28 | 0.154 |
| Hydrochloric Acid | 0.05 | 0.45 | 0.45 | 0.05 | 1.28 | 0.150 |
| Itaconic | 0.16 | 0.33 | 0.44 | 0.07 | 1.24 | 0.022 |
| Oxalic Acid | 0.11 | 0.38 | 0.44 | 0.07 | 1.24 | 0.182 |
| D-glucuronic | 0.24 | 0.25 | 0.44 | 0.07 | 1.24 | 0.0003 |
| Acid | | | | | | |
| D-galacturonic | 0.24 | 0.25 | 0.44 | 0.07 | 1.24 | 0.0003 |
| Acid | | | | | | |
| Ribonolactone | 0.18 | 0.31 | 0.44 | 0.07 | 1.25 | 0.0001 |
| D-gulono-1,4- | 0.22 | 0.27 | 0.43 | 0.07 | 1.26 | 0.0002 |
| lactone | | | | | | |
| L-gulono-γ- | 0.22 | 0.27 | 0.44 | 0.07 | 1.25 | 0.0001 |
| lactone | | | | | | |
| α-glucoheptonic | 0.26 | 0.24 | 0.44 | 0.07 | 1.24 | 0.0001 |
| lactone | | | | | | |
| Acetic Acid | 0.07 | 0.40 | 0.45 | 0.07 | 1.23 | 0.0006 |
| Butyric Acid | 0.11 | 0.38 | 0.44 | 0.07 | 1.21 | 0.0007 |

### Liamocin Conversion in Flow Reactor

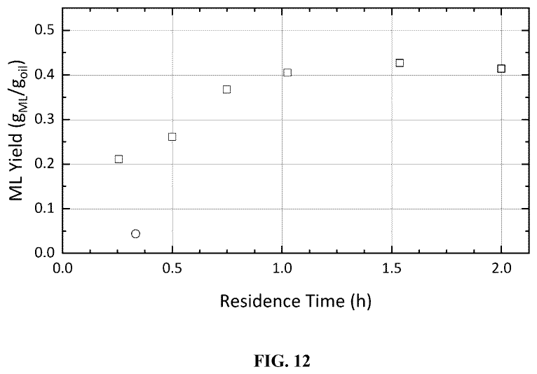

High temperature/pressure hydrolysis-dehydration reactions were performed in a custom-built flow reactor which consisted of a quarter inch outer diameter stainless-steel tube of known volume (10 or 25 mL), two Shimadzu HPLC pumps for supplying feeds, an Agilent 6890 GC for heating the reactor, and an Idex 500 psi backpressure regulator. To minimize the conversion outside the reactor all tubing before and after the reactor was 0.02-inch internal diameter to minimize the internal volume. For all flow reactions the feeds were 10 wt % liamocin oil in MEK and 50 wt % citric acid in water. The densities of the organic and aqueous feeds were determined to be 0.805 and 1.258 g/mL, respectively, and the pump flow rates (mL/min) were set to provide equal mass flow rates of the two feeds. For each experiment the reactor was allowed to come to steady state over three residence times before collecting samples for analysis. Massoia lactone production was monitored by HPLC using the same method as the batch reactions. Results for reactions performed in the flow reactor at 500 psi and either 120° C. or 150° C. are shown in FIG. 12. At 120° C. and a residence time of 20 min a ML yield of 0.043 g/gₒᵢₗ was obtained, similar to what was obtained in 6 h at 70° C. in the batch reactions (FIG. 5, triangles) when similar solvent/reactant/catalyst concentrations were used. Increasing the reaction temperature to 150° C. resulted in higher ML yields. A ML yield of 0.22 g/gₒᵢₗ was achieved at a residence time of 0.15 h, 1.45× higher than was seen at 70° C. after 24 h (Table 1). ML yields increased with longer residence times and reached a value of 0.47 g/gₒᵢₗ at a residence time of 1.5 h. These results for the unoptimized reaction correspond to 65% of the estimated maximum yield of ML when it is assumed 2.5 dihydroxydecanoic acids/liamocin.

A batch distillation was performed on the flow reactor effluent produced at 150° C. and a residence time of 2 hr. The effluent was charged into a three neck round bottom flask along with a Teflon stir bar. A thermocouple was inserted above the liquid through one of the side necks to monitor the pot temperature and the other was sealed with a rubber septum. A still head with a jacketed condenser was connected to the vertical neck and a second thermocouple was inserted through an adapter into the still head monitor vapor temperatures in the head. Heat was provided using a heating mantle powered with a Variac variable voltage transformer and the condenser temperature was controlled at 10° C. with a circulating water chiller. Distillate fractions were collected for 5 min. Over the course of 4 collected fractions the temperature of the vapors in the pot rose from 72° C. to 98° C. while the head temperature was essentially constant at 72° C. When the head temperature began to increase the distillation was stopped and cooled to room temperate. HPLC analysis of the distillate fractions showed that MEK was the primary component along with water and there was no observed Massoia lactone product. Upon cooling the residual liquid in the pot separated into two phases that were transferred to a separatory funnel for recovery. HPLC analysis on the top organic layer was consistent with Massoia lactone and no MEK or citric acid were observed. Analysis of the aqueous fraction showed all the MEK was removed from the pot during distillation. These results demonstrate that the Massoia lactone product can be easily recovered from the reaction mixture by removal of the MEK solvent.

While the invention has been described with reference to details of the illustrated embodiments, these details are not intended to limit the scope of the invention as defined in the appended claims. The embodiment of the invention in which exclusive property or privilege is claimed is defined as follows:

What is claimed is:

1. A method of producing Massoia lactone, comprising the steps of:
preparing a reaction mixture comprising water, a carboxylic acid, methyl ethyl ketone, and a liamocin; and
incubating the reaction mixture under conditions allowing for the conversion of the liamocin to Massoia lactone.

2. The method of claim 1, wherein the ratio of water to carboxylic acid is about 20:80 to about 80:20.

3. The method of claim 1, wherein the carboxylic acid is succinic acid, malic acid, tartaric acid, citric acid, or a combination thereof.

4. The method of claim 1, wherein the reaction mixture further comprises an exophilin.

5. The method of claim 4, wherein the conditions also allow for the conversion of the exophilin to Massoia lactone.

6. The method of claim 1, wherein the water and carboxylic acid are combined prior to adding to the reaction mixture.

7. The method of claim 1, wherein the methyl ethyl ketone and liamocin are combined prior to adding to the reaction mixture.

8. The method of claim 1, wherein the liamocin comprises an Aureobasidium pullulans culture filtrate.

9. The method of claim 1, wherein the incubating step is performed for at least about one hour.
